(19) United States
(12) Patent Application Publication
(10) Pub. No.: US 2014/0323995 A1
(43) Pub. Date: Oct. 30, 2014

(54) Targeted Drug Delivery Devices and Methods

(71) Applicant: Transcend Medical, Inc., Menlo Park, CA (US)

(72) Inventors: Luke Clauson, Menlo Park, CA (US); Tsontcho Ianchulev, Menlo Park, CA (US); Nathan White, Menlo Park, CA (US); Richard S. Lilly, Menlo Park, CA (US); Matthew Newell, Menlo Park, CA (US); Michael Schaller, Menlo Park, CA (US)

(73) Assignee: TRANSCEND MEDICAL, INC., Menlo Park, CA (US)

(21) Appl. No.: 14/260,041

(22) Filed: Apr. 23, 2014

Related U.S. Application Data
(60) Provisional application No. 61/815,681, filed on Apr. 24, 2013.

(51) Int. Cl. A61F 9/00

(52) U.S. Cl. CPC A61F 9/0017
USPC 604/290; 604/294

(57) ABSTRACT

This disclosure relates generally to methods and devices for use in treating eye conditions. In some embodiments, a site-specific therapeutic agent is mixed with a releasing agent with a dual syringe apparatus in order to achieve homogeneity. Once mixed, the site-specific therapeutic agent and releasing agent can be either dispensed directly within an area of the eye or within an implant. The implant can be at least partially filled with the site-specific therapeutic agent and releasing agent either prior to or after implantation into the eye. Some ratios of site-specific therapeutic agents to releasing agents are disclosed which provide various releasing profiles of the site-specific therapeutic agent within the eye.

### CROSS-REFERENCE TO RELATED APPLICATION

This application claims priority to U.S. Provisional Patent Application Ser. No. 61/815,681, titled “Targeted Drug Delivery Devices and Methods,” filed Apr. 24, 2013, the disclosure of which is hereby incorporated by reference herein. Priority of the aforementioned filing date is claimed.

### BACKGROUND

This disclosure relates generally to methods and devices for use in treating eye conditions with drug and therapeutic agents either delivered directly into the eye or from an implantable drug delivery device. The mechanisms that cause glaucoma are not completely known. It is known that glaucoma results in abnormally high pressure in the eye, which leads to optic nerve damage. Over time, the increased pressure can cause damage to the optic nerve, which can lead to blindness. Treatment strategies have focused on keeping the intraocular pressure down in order to preserve as much vision as possible over the remainder of the patient's life. Various drugs and therapeutic agents can assist in both the treatment of ocular diseases, including glaucoma.

The bioavailability of at least some ophthalmic drugs can be poor due to efficient protective mechanisms of the eye. In addition, anatomical features, physiology and chemical properties of the eye can make targeted delivery of drugs challenging. Protective barriers such as blinking, baseline and reflex lachrymation, conjunctival absorption and drainage can rapidly remove drugs which have been delivered to the eye. Additionally, the heterogenous nature of the cornea can pose a significant challenge for topical applications of pharmaceuticals. Therefore, it can be beneficial to circumvent or overcome at least some protective barriers of the eye without causing stress or permanent damage to the eye.

### SUMMARY

The subject matter described herein provides many advantages. For example, the current subject matter includes improved therapeutic agents, devices and methods for the treatment of the eye.

Disclosed herein are devices and methods for delivering a therapeutic agent into the eye. An embodiment of a method includes filling a first syringe of a dual syringe apparatus with the therapeutic agent and filling a second syringe of the dual syringe apparatus with a releasing agent. In addition, the method can include coupling the first syringe to the second syringe and mixing the therapeutic agent with the releasing agent by pushing on at least one plunger of the dual syringe apparatus. Additionally, the method can include dispensing the therapeutic agent mixed with the releasing agent into at least one of a part of the eye or an ocular implant.

More details of the devices, systems and methods are set forth in the accompanying drawings and the description below. Other features and advantages will be apparent from the description and drawings.

### BRIEF DESCRIPTION OF THE DRAWINGS

These and other aspects will now be described in detail with reference to the following drawings. Generally speaking the figures are not to scale in absolute terms or comparatively but are intended to be illustrative. Also, relative placement of features and elements may be modified for the purpose of illustrative clarity

FIG. 1 is a cross-sectional, perspective view of a portion of the eye showing the anterior and posterior chambers of the eye.

FIG. 2 is a cross-sectional view of a human eye.

FIGS. 3A-3B show embodiments of drug delivery devices being used to treat a condition of the eye.

FIG. 4A shows an embodiment of a drug delivery device having shape memory in a delivery conformation.

FIGS. 4B-4D show top, side and perspective views, respectively of the drug delivery device of FIG. 4A in an implantation conformation.

FIGS. 5A-5B show an embodiment of an implant filled with a drug-release material.

FIGS. 6A-6D show variations of a delivery tool for delivering an implant(s) into the eye.

FIG. 7 shows a delivery tool being used to deliver an implant into the eye.

FIG. 8 shows another embodiment of an implantation system for delivery of an implant.

FIGS. 9A-9D shows the implantation system of FIG. 9 filling an implant with a flowable material upon delivery in the eye.

FIG. 10 shows a schematic view of distal deposition of a flowable material near a distal end of an implant.

FIG. 11 shows a schematic view of a cross-sectional view of the eye having an implant and a distal deposition creating a lake within the surrounding tissues.

FIG. 12 shows an embodiment of a guidewire delivering site-specific therapeutic agents to a sub-retinal space within the eye.

FIG. 13 shows an embodiment of a dual syringe assembly.

FIGS. 14A-14C show embodiments of a connecting element of the assembly.

FIG. 15 shows an alternate embodiment of a connecting element.

Like reference symbols in the various drawings indicate like elements.

### DETAILED DESCRIPTION

Described herein are devices, systems and methods for the treatment of eye diseases such as glaucoma, macular degeneration, retinal disease, proliferative vitreoretinopathy, diabetic retinopathy, uveitis, keratitis, cytomegalovirus retinitis, cystoid macular edema, herpes simplex viral and adenoviral infections and other eye diseases. The devices described herein can deliver therapeutics to select regions and structures. The devices described herein can deliver therapeutics in a time-release fashion within the eye. The devices described herein can include memory devices that change shape upon implantation as will be described in more detail below. The implants described herein can include a drug-release material such as a biodegradable polymer impregnated with a drug, wherein the drug can be delivered in a time-release fashion and used for disease treatment such as reduction of aqueous production or improved outflow of aqueous through uveoscleral structures or the treatment of other eye disorders

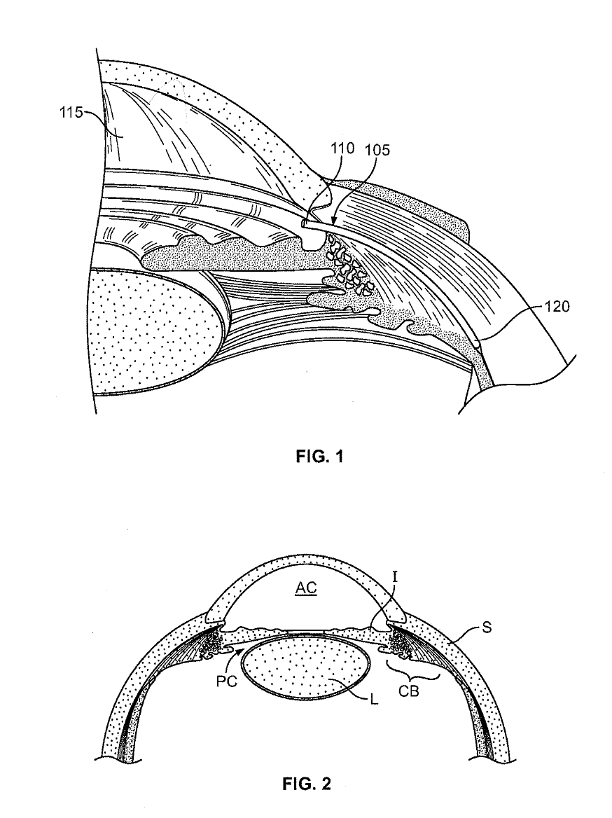

FIG. 1 is a cross-sectional, perspective view of a portion of the eye showing the anterior and posterior chambers of the eye. A schematic representation of an implant 105 is positioned inside the eye such that a proximal end 110 is located in the anterior chamber 115 and a distal end 120 is located in or near the suprachoroidal space (sometimes referred to as the perichoroidal space). The suprachoroidal space can include the region between the sclera and the choroid. The suprachoroidal space can also include the region between the sclera and the ciliary body. In this regard, the region of the suprachoroidal space between the sclera and the ciliary body may sometimes be referred to as the supraciliary space. The suprachoroidal “space” is a potential space between tissue layers that does not normally exist physiologically or histologically. Rather, the suprachoroidal space can be artificially created such as by surgical methods and devices such that an implant or other material can be implanted therein.

The implants 105 described herein can deliver therapeutics to the eye in a tailored manner. For example, a single implant can deliver a single therapeutic to a single region of the eye. Alternatively, a single implant can deliver more than one therapeutic to a region of the eye by incorporating drug delivery zones. Further, multiple implants can be delivered to multiple regions of the eye to deliver one or more therapeutics to those regions. It should also be appreciated that the implants described herein are not necessarily positioned between the choroid and the sclera. The implants can be positioned at least partially between the ciliary body and the sclera, or at least partially positioned between the sclera and the choroid. The implants described herein can also be implanted such that they extend towards the back of the eye and other regions in the eye as will be described herein.

FIG. 2 is a cross-sectional view of a portion of the human eye. The eye is generally spherical and is covered on the outside by the sclera S. The retina lines the inside posterior half of the eye. The retina registers the light and sends signals to the brain via the optic nerve. The bulk of the eye is filled and supported by the vitreous body, a clear, jelly-like substance. The elastic lens L is located near the front of the eye. The lens L provides adjustment of focus and is suspended within a capsular bag from the ciliary body CB, which contains the muscles that change the focal length of the lens. A volume in front of the lens L is divided into two by the iris I, which controls the aperture of the lens and the amount of light striking the retina. The pupil is a hole in the center of the iris I through which light passes. The volume between the iris I and the lens L is the posterior chamber PC. The volume between the iris I and the cornea is the anterior chamber AC. Both chambers are filled with a clear liquid known as aqueous humor.

The ciliary body CB continuously forms aqueous humor in the posterior chamber PC by secretion from the blood vessels. The aqueous humor flows around the lens L and iris I into the anterior chamber AC and exits the eye through the trabecular meshwork, a sieve-like structure situated at the corner of the iris I and the wall of the eye (the corner is known as the iridocorneal angle). Some of the aqueous humor filters through the trabecular meshwork near the iris root into Schlemm's canal, a small channel that drains into the ocular veins. A smaller portion rejoins the venous circulation after passing through the ciliary body and eventually through the sclera (the uveoscleral route).

Glaucoma is a disease wherein the aqueous humor builds up within the eye. In a healthy eye, the ciliary processes secrete aqueous humor, which then passes through the angle between the cornea and the iris. Glaucoma appears to be the result of clogging in the trabecular meshwork. The clogging can be caused by the exfoliation of cells or other debris. When the aqueous humor does not drain properly from the clogged meshwork, it builds up and causes increased pressure in the eye, particularly on the blood vessels that lead to the optic nerve. The high pressure on the blood vessels can result in death of retinal ganglion cells and eventual blindness.

Closed angle (acute) glaucoma can occur in people who were born with a narrow angle between the iris and the cornea (the anterior chamber angle). This is more common in people who are farsighted (they see objects in the distance better than those which are close up). The iris can slip forward and suddenly close off the exit of aqueous humor, and a sudden increase in pressure within the eye follows.

Open angle (chronic) glaucoma is by far the most common type of glaucoma. In open angle glaucoma, the iris does not block the drainage angle as it does in acute glaucoma. Instead, the fluid outlet channels within the wall of the eye gradually narrow with time. The disease usually affects both eyes, and over a period of years the consistently elevated pressure slowly damages the optic nerve.

It should be appreciated that other ocular conditions besides glaucoma can be treated with the implants described herein. For example, the implants can deliver drugs for the treatment of macular degeneration, retinal disease, proliferative vitreoretinopathy, diabetic retinopathy, uveitis, keratitis, cytomegalovirus retinitis, cystoid macular edema, herpes simplex viral and adenoviral infections. It also should be appreciated that medical conditions besides ocular conditions can be treated with the implants described herein. For example, the implants can deliver drugs for the treatment of inflammation, infection, cancerous growth. It should also be appreciated that any number of drug combinations can be delivered using any of the implants described herein.

In a first embodiment, the implant 105 can have a solid body that does not include a flow channel such that agents are delivered into the eye by the drug delivery implant independent of a flow channel. The implant 105 can be an elongate element having a substantially uniform diameter along its entire length as shown in FIG. 1. It should be appreciated, however, that the implants can vary widely in shape, structure and also material as will be described in more detail below. Moreover, the implant 105 can have various cross-sectional shapes (such as a, circular, oval or rectangular shape) and can vary in cross-sectional shape moving along its length. The shape of the implant 105 can also vary along its length (either before or after insertion of the implant). The cross-sectional shape can be selected to facilitate easy insertion into the eye. The implant 105 can be formed at least in part by a material having shape memory, such as a shape memory metal alloy, such as Nitinol, or a heat-set polymer. The implant 105 can transition from a narrow, elongate delivery shape to its memory shape upon delivery in the eye. For example, the elongate implant can relax into a shape that is curved, coiled, cupped, rolled, twisted, tangled and the like.

The implant can have a thin, elongated structure, such as a fiber, filament or a monofilament wire of polymer. The filamentous implant can also include a plurality of interconnected strands, such as in a twist or braid or other woven fashion. The filamentous implant can also take on a tangled configuration that resembles a tangled ball of string. The implant can also have a shorter structure such as segments of fibers, or spherical particles such as pellets, beads or deposits of polymer, gel or other material. The implant can have a structure that includes a body having an inner core that can be filled with an agent to be delivered, such as a “pumping pill” type of implant, as will be described in more detail below. The implant can include one or more nanotubes.

The implant 105 can include a drug-eluting polymer matrix that is loaded or impregnated with a drug. The drug can elute over time into the eye from the implant 105 in a time-release fashion. The implant 105 or a portion of the implant can be bioabsorbable such that it need not be removed from the eye after administration of the drug protocol. The implant 105 or a portion of the implant can also be non-bioabsorbable as well. The non-bioabsorbable implant can, but need not be removed from the eye once the drug is fully administered. If the implant is to be removed from the eye upon final delivery of drug, the removal and replacement schedule can vary. For example, the implant can be removed and replaced every 1-2 years. The implant 105 can include a feature such as a proximal loop or other structure that can be grasped allowing the implant 105 to be retrieved and replaced. A portion of the implant 105 can also be anchored, for example with structural features such as flanges, protrusions, wings, tines, or prongs, and the like that can lodge into the surrounding eye anatomy to retain its position during drug delivery.

As mentioned above, the implants described herein can be positioned within a variety of regions within the eye including the supraciliary space, suprachoroidal space, and further back towards the back of the eye. The suprachoroidal space (sometimes referred to as the perichoroidal space) can include the region between the sclera and the choroid. The suprachoroidal space can also include the region between the sclera and the ciliary body. In this regard, the region of the suprachoroidal space between the sclera and the ciliary body may sometimes be referred to as the supraciliary space. For example, the implants described herein can be positioned within different regions of the eye depending on the condition to be treated. An implant being used to deliver a drug used to treat macular degeneration, for example, can be positioned such that at least a portion of the implant is positioned near the back of the eye. An implant being used to deliver an anti-glaucoma drug can be positioned, for example, within at least a portion of the supraciliary and/or suprachoroidal space.

The implants described herein can also deliver one or more therapeutics to select regions and structures within the eye by the formulation of one or more drug delivery zones along the length of the implant. In an embodiment, the implant can be coated on a surface with one or more drugs to create the one or more drug delivery zones. The implants can include one, two, three, or more drug delivery zones. Each drug delivery zone can deliver one or more drugs. The drug delivery zones can be formulated depending on where the zone is oriented within the eye upon implantation of the device. Orientation of the drug delivery zones with respect to the adjacent tissues can be selected based on where drug delivery is desired. For example, drugs that affect outflow of aqueous, for example through the trabecular meshwork can be embedded or delivered from a drug delivery zone positioned in the anterior chamber, near the trabecular meshwork, iris, Schlemm's canal and the like. Drugs that affect production of aqueous from epithelial cells of the ciliary body can be can be embedded or delivered from a drug delivery zone positioned near the ciliary body, the epithelial cells of the ciliary body, the boundary between the ciliary body and the sclera, the supraciliary space, the suprachoroidal space and the like.

The implant can be implanted such that one drug delivery zone is positioned in a first anatomical location, for example between the ciliary body and the sclera, and the other drug delivery zone is positioned in a second anatomical location. The type of drug delivered from each drug delivery zone can be site-specific therapeutic agents such that they are tailored to where in the eye anatomy the drug delivery zone is positioned. Zones positioned between the ciliary body and the sclera can contain drug(s) that affect the ciliary body, for example, a drug that acts on the ciliary body epithelial cells to decrease aqueous humor production. This tailored formulation of the drug delivery zones allows for a direct route of administration to intended drug targets within the eye. Drug dosage can be reduced compared to, for example, systemic delivery or for avoiding problems with wash-out. The implant as well as each drug delivery zone relative to the implant can have a length that is suitable for desired delivery of a drug in and around various structures within the eye.

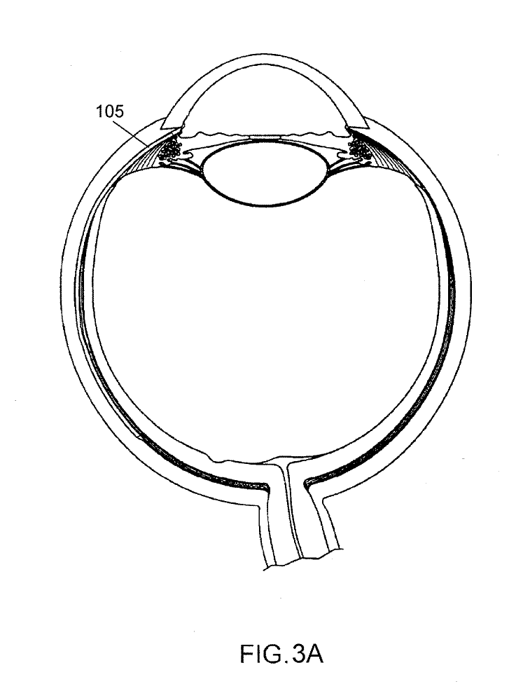

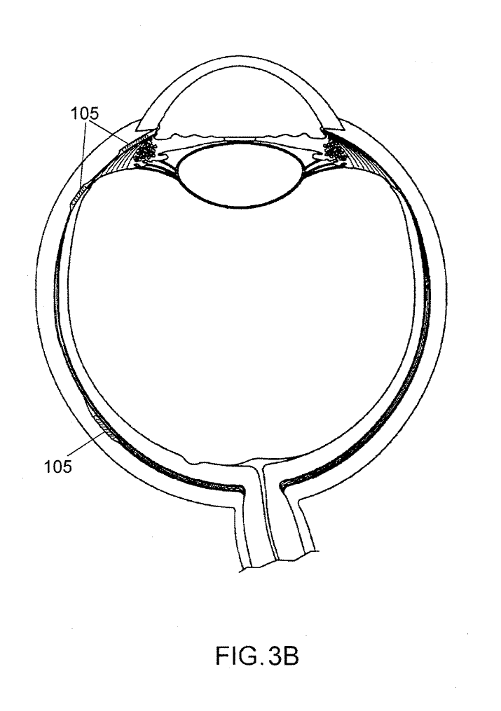

FIG. 3A shows an embodiment of a drug delivery implant 105 that has an elongate, filamentous structure and extends between the region of the eye near the ciliary body towards the back of the eye. The implant 105 can include one or more drug delivery zones which can provide one or more site-specific therapeutic agents depending on which anatomical location of the eye is desired to be treated. More than one disease or condition can be treated from a single implant. For example, both retinal disease and glaucoma can be treated from one implant. It should also be appreciated that the number of drug delivery zones can vary and that different medications can be used to treat different portions of the eye in the different zones of the implants.

The elongate, filamentous structure can be delivered such that it trails through multiple locations in the eye as shown in FIG. 3A. For example, the distal end of a single filamentous implant can be dragged into place to a location near the back of the eye while the proximal end remains positioned near the ciliary body. The implant having an elongate, filamentous structure can be delivered such that it takes on a different structure. For example, a filamentous implant can be delivered such that it bunches or tangles up within a focused region in the eye. The elongate, filamentous implant can also be manufactured of a material having shape memory that changes from a delivery conformation to an implantation conformation, as will be discussed in more detail below.

In addition to using an elongate implant having multiple drug delivery zones to tailor drug treatments, more than one implant 105 can be positioned in multiple locations within the eye (see FIG. 3B). Multiple implants 105 can be used to treat more than one condition or the multiple implants can treat a single condition by delivering one or more therapeutic agents. Multiple pellets of drug delivery polymer or gel impregnated with a therapeutic can be delivered in single or multiple locations in the eye. The implants delivered to multiple locations can include segments of fibers, or spherical particles such as pellets, beads or deposits of polymer, gel or other material.

The dimensions of the implants can vary. In an embodiment, the implant has a length in the range of about 0.1″ to about 0.75″. In another embodiment, the implant as a length of about 0.250″ to about 0.300″. In another embodiment, the implant as a diameter in the range of about 0.002″ to about 0.015″. In another embodiment, the implant has a diameter in the range of about 0.002″ to about 0.025″. In an embodiment, the diameter if the implant is 0.012″, 0.010″, or 0.008″. In the event that multiple implants are used, each implant can be about 0.1″. Stacking the implants can result in a fully implanted device having a length, for example of 0.2″ to 1.0″, although the length can be outside this range. An embodiment of the implant is 0.250″ long, and 0.015″ in outer diameter. One embodiment of the implant is 0.300″ long. In another embodiment, the implant is approximately 1 mm in diameter and between about 15-20 mm in length. In another embodiment, the implant is approximately 1 mm in diameter and approximately 3 mm in length. In another embodiment, the implant is approximately 1 mm².

Depending on the treatment dose desired, and the delivery profile of the therapeutic agent delivered, it may be advantageous for the implant 105 to extend from the initial dissection plane near the angle of the eye, within the supraciliary and/or suprachoroidal space into the posterior segment of the eye or any location therebetween. The geometry of the implant 105 can assist in the ability to prolong or control various dosing regimes. For example, a longer implant 105, multiple implants 105 or an implant 105 having a larger diameter can each result in a longer dosing potential. The implant 105 can completely fill the suprachoroidal space to minimize any “washout” effect as well as assist in the dosing. In addition, it may be advantageous to employ a sealant, to seal any communication between the anterior chamber and the newly dissected suprachoroidal space once the implant 105 is placed. Products such as TISSEAL (Baxter Healthcare, Irvine, Calif.), fibrin glues, or small amounts of cyanoacrylate may be used for this purpose.

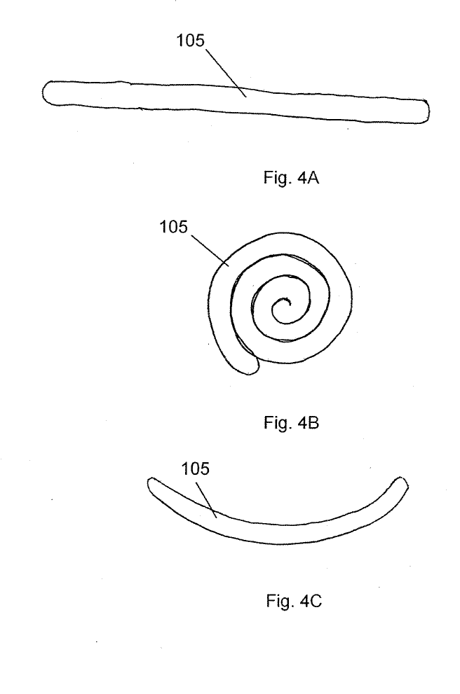

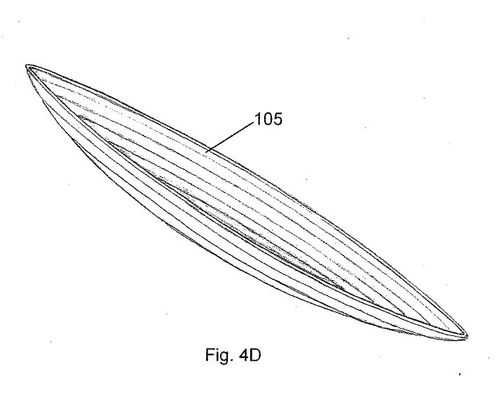

As mentioned above, the elongate, filamentous implant can also be manufactured of a material having shape memory, such as a heat-set polymer, Nitinol or other shape-memory alloy that changes from a delivery conformation to an implantation conformation. The implant 105 can change from a delivery conformation such as that shown in FIG. 4A to an implantation conformation such as those shown in FIGS. 4B-4D. The implant 105 upon being released in the eye can take on its relaxed shape such as a coil. The coil can also take on a cup shape (see FIGS. 4C and 4D) such that it hugs the curve of the eye and minimizes distortion of surrounding eye tissues, for example the retina if implanted near the back of the eye or the zonules if implanted near the ciliary body.

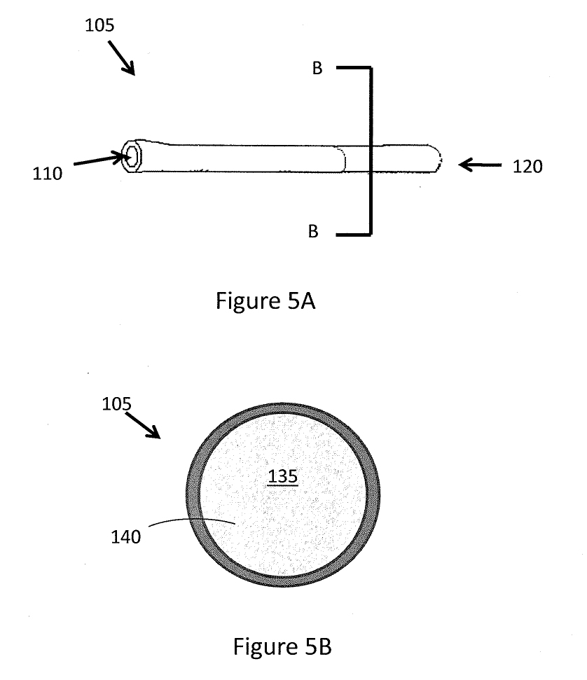

FIG. 5A shows another embodiment of an implant 105 and FIG. 3B is a cross-sectional view of the implant of FIG. 3A taken along lines B-B. In this embodiment, the implant 105 can be an elongate element having one or more interior volumes 135 into which a drug-release material 140 can be molded, cast, embedded or injected therein, as will be described in more detail below. In an embodiment, the drug-release material 140 plugs the interior volume(s) 135 and prevents fluid flow through the implant for a period of time. An amount of drug within the drug-release material 140 can elute over time from the interior volume 135, for example through an opening in fluid communication with the interior volume 135, to treat a region of the eye. After a period of time, the drug-release material 140 degrades and is removed from the interior volume(s) 135 of the implant, as will be discussed in more detail below. Alternately, the drug release material can be nondegradable. The drug and/or a drug-release material can degrade out of a non-absorbable structure, leaving the interior volume only including a matrix of the non-absorbable structure.

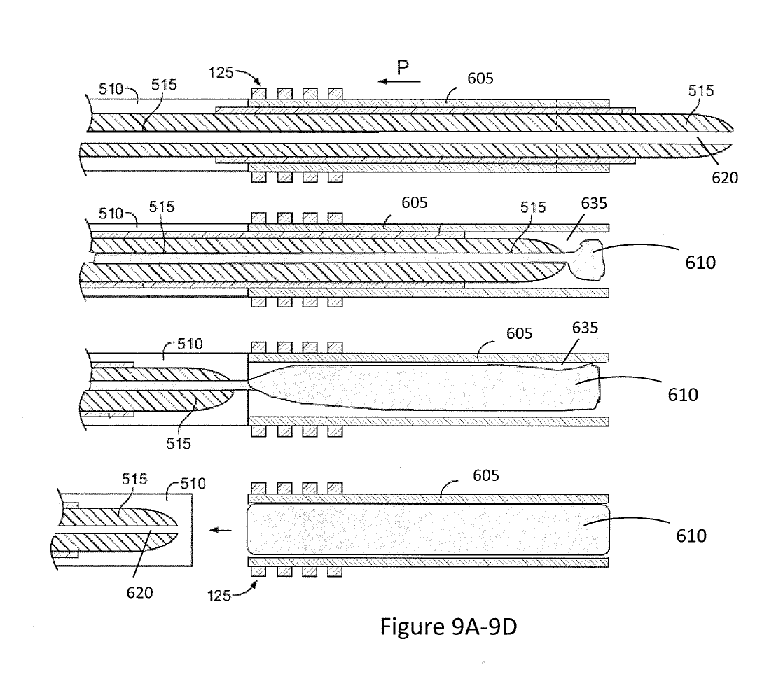

As described with previous embodiments, the implant 105 can have a substantially uniform diameter along its entire length, although the shape of the implant 105 can vary along its length as described above. The cross-sectional shape can be selected to facilitate easy insertion into the eye. The implant 105 can include any number of additional structural features 125 that aid in anchoring or retaining the implanted implant 105 in the eye (see FIGS. 9A-9D) such as protrusions, wings, tines, or prongs that lodge into anatomy to retain the implant in place. In an embodiment, the interior volume 135 can also be used as a pathway for flowing material (for example, aqueous, liquid, balanced salt solution, viscoelastic fluid, therapeutic agents, drug-release material, or the like) into the eye. U.S. Patent Publication Nos. 2007-0191863 and 2009-0182421 describe exemplary implants. These applications are incorporated by reference in their entirety.

In the embodiment of FIGS. 5A-5B, the implant 105 can include an interior volume 135 extending between at least one opening 110 at a proximal end and at least one opening 120 at a distal end. The interior volume 135 can be filled with a drug-release material 140 forming a plug that can prevent a substantial flow of fluid through the implant 105. The implant 105 having drug-release material 140 in the internal volume 135 which can serve as a drug delivery implant to deliver therapeutics in a time-release fashion to the anterior chamber, the suprachoroidal space or other regions near the eye. In an embodiment, the drug is completely eluted from the drug-release material 140 over a selected period of time. Further, the drug-release material 140 can degrade over another selected period of time such that it no longer plugs the interior volume 135. As such, some flow can begin to take place through the interior volume 135 in the implant 105.

In addition, the interior volume 135 can be filled with a site-specific therapeutic agent within the drug release material. In such an embodiment, the site-specific therapeutic agent can be delivered to one or more specific anatomical tissues or features within the eye. For example, the interior volume 135 can include one or more of an anti-fibrotic agent, such as 5FU or MMC, or anti-inflammatory. In addition, the site-specific therapeutic agent, such as either the anti-fibrotic agent or anti-inflammatory, can be mixed within a viscoelastic material, such as hyaluronic acid. The viscoelastic material can assist in controlling the kinetics of release of the site-specific therapeutic agent. Additionally, the ratio of viscoelastic to site-specific therapeutic agent can assist in varying the kinetics of release of the site-specific therapeutic agent into the eye. For example, the ration of the viscoelastic material to site-specific therapeutic agent can allow the kinetics of release of the site-specific therapeutic agent to release as a burst or over a long period of time, such as one or more weeks. As will be discussed, a variety of ratios of viscoelastic to site-specific therapeutic agents are disclosed herein and the ratios of viscoelastic to site-specific therapeutic agents can be delivered into the eye with or without the assistance of an ocular implant.

The walls of the implant 105 can have a solid structure or can include one or more openings extending from an internal surface to the external surface through which the drug-release material 140 can elute. The implant 105 can also have a braided or mesh structure such that the openings in the braided or mesh structure are spanned, or partially spanned, by drug-release material 140. The implant 105 can include one or more internal reservoir(s) of drug that fluidly communicate with the surface of the implant such that drug-release material 140 can elute from the reservoirs and come into contact with adjacent tissues. The reservoirs can be refillable and/or a single-use reservoir. The reservoirs can be opened such as by a laser or other energy source to apply a small electrical voltage to release the desired dose of the drug(s) on demand.

The implants 105 described herein can deliver more than one type of drug simultaneously, including site-specific therapeutic agents. In an embodiment, the implant 105 can include a second drug, which may be incorporated into the drug-release material, the implant itself or both. The implant 105 can release one, two, three, four or even more drugs. The drug-release material 140 can include more than a single therapeutic. Alternatively, the drug-release material 140 can be divided into drug delivery zones, such as one, two, three, or more drug delivery zones within or on the implant 105. For example, a distal end of the implant can include a first zone of drug-release material 140 and a proximal end of the implant can include a second zone of drug-release material 140 that elutes a different drug. Further, each drug delivery zone can deliver one or more drugs. Implants having drug delivery zones are described in more detail in application Ser. No. 12/939,033, filed Nov. 3, 2010, which is incorporated herein by reference in its entirety. The implants 105 described herein can also have one or more coatings or be covered by one or more films. The implant 105 can be coated with one or more surface layers of materials, such as a slow-release substance to have prolonged effects on local tissue surrounding the implant 105. As such a material can be released from the surface of the implant and a different material can be released from the interior of the implant.

As mentioned above, the implants described herein can, but need not be removed from the eye upon completion of a drug delivery protocol. If the implant is to be removed from the eye upon final delivery of drug, the removal and replacement schedule can vary. For example, the implant can be removed and replaced every 1-2 years. Alternatively, the implants can be left within the eye after full elution of drug from the drug-release material and degradation of the drug-release material from the interior volume. In an embodiment, the implant can be biodegradable and need not be removed from the eye after administration of the drug protocol. The biodegradable material selected for the implant body can have a similar or longer degradation rate than the drug-release material 140 within the core of the implant 105 or spanning the openings of the implant 105, but will generally have a longer degradation rate than the elution rate of the drug from the drug-release material, as will be discussed in more detail below.

As used herein, “drug-release,” “drug-eluting,” “drug-loaded” materials and the like refer to materials that are or can have a substance such as a drug or therapeutic agent, including site-specific therapeutic agents, dissolved, entrapped, encapsulated, loaded, impregnated, adsorbed, or otherwise embedded within the material for controlled delivery of the substance into tissues. It should be appreciated that use of the term “drug” is not limiting regarding what substance is admixed with the drug-release material. The drug-release material can include essentially any biocompatible polymer, co-polymer, terpolymer, polymer blend, as well as non-polymeric substances and matrices. The drug-release material can include biodegradable materials including bioerodible, bioabsorbable, and bioresorbable polymeric materials. Examples of non-polymeric materials that can be employed include, but are not limited to, metal oxide structures, metallic matrices and other porous substances. The drug-release material can be designed as blends, films, matrices, microspheres, nanoparticles, pellets, coatings, films, cores etc.

The drug-release material can be biodegradable polymers including, but not limited to poly(lactic-co-glycolic) acid (“PLGA”), polylactide, polyglycolide, polycaprolactone, or other polyesters, poly(orthoesters), poly(aminoesters), polyanhydrides, polyorganophosphazenes, or any combination thereof. Other biodegradable polymers known to those skilled in the art may also be applied and selected based on the desired mechanical properties and polymer-drug interaction.

In another embodiment, the polymer of the drug-release material is non-degradable. For example, the polymer of the drug-release material may be ethyl cellulose, poly(butyl acrylate), poly(urethanes), silicone resins, nylon, ammonium polyacrylate, acrylamide copolymers, acrylate/acrylamide copolymers, acrylate/ammonium acrylate copolymers, acrylate/alkyl acrylate copolymers, acrylate/carbamate copolymers, acrylate/dimethylaminoethyl methacrylate copolymers, ammonium acrylate copolymers, styrene/acrylate copolymers, vinyl acetate/acrylate copolymers, aminomethylpropanol/acrylate/dimethylaminoethylmethacrylate copolymers, or any combination thereof. Other non-degradable polymers known to those skilled in the art may also be applied and selected based on the desired mechanical properties and polymer-drug interaction.

In some embodiments, the drug-release material can include a hydrogel, including, but not limited to, polyhydroxyethylmethacrylate (pHEMA), a silicone, agarose, alginate, chitosan, and hyaluronic acid. The drug-release material can also include a viscoelastic composition such as a viscoelastic preparation of sodium hyaluronate such as AMVISC (from Anika Therapeutics, Inc.), OCUCOAT (Bausch & Lomb), PROVIS, VISCOAT, DUOVISC, CELLUGEL (from Alcon Labs), BIOVISC, VITRAX (from Allergan), BIOLON (from Bio-Technology General), STAARVISC (from Anika Therapeutics/Staar Surgical), SHELLGEL (from Anika Therapeutics/Cytosol Opthalmics), HEALON (Abbott Medical Optics), UNIVISC (from Novartis), and the like. Other hydrogels known to those skilled in the art may also be applied and selected based on the desired mechanical properties and hydrogel-drug interaction. The drug-release material may, in some cases, form a gel within a pH range. In another embodiment, the drug-release material may transition between a liquid and a gel at a critical temperature. In another embodiment, a physical or chemical interaction between the hydrogel or viscoelastic can be employed to regulate the drug release rate.

Release of the drug from the drug-release material can be controlled, in part, by the composition of the polymer in the drug-release material. Various factors such as the mechanical strength, swelling behavior, capacity to undergo hydrolysis all can affect release rates of the drug-release material, as is known in the art. The polymer can be engineered and specifically designed and/or selected to provide the drug-release material with the desired biodegradation rate and release profile of the drug from for a selected duration. The release profile can be manipulated such as by adjusting features of the composition like polymer(s), changing the ratio of components of the polymeric material, ratio of the monomers in the co-polymer drug(s), level of drug loading, surface area and dimensions of the implant etc. The ratio of polymer, or drug-release material, to drug can vary as well. For example, the polymer, or drug-release material, to drug ratio can include 1:1, 2:3, 1:3, 1:6 1:2, 1:4, 1:8, 1:16, 1:32, 1:64, 1:128, 1:256, 1:512, or any other desirable ratio. In addition, the polymer, or drug release material, to drug ration can include 6:1, 3:1, 2:1, and 3:2. In an embodiment, the ratio of therapeutic agent to releasing agent is between 1:1 and 1:8.

The drug-release material can release a drug, including a site-specific therapeutic agent, over a period of time. In an embodiment, the drug-release material releases at least one drug for at least 12 hours, at least 18 hours, at least 24 hours, at least 48 hours, at least 3 days, at least 7 days, at least 14 days, at least 30 days, at least 60 days, at least 90 days, at least 100 days, at least 120 days, at least 150 days, at least 180 days, at least 200 days, at least 250 days, at least 300 days, at least 350 days, at least 400 days, or even longer.

The drug-release material can exhibit multi-phasic drug release profiles, which can include an initial burst of drug and a period of sustained drug release as is known in the art. In addition, some release profiles of the drug-release material, including the site-specific therapeutic agent, can include a constant rate or exponential rate of release profile. The release profile can be manipulated such as by adjusting features of the composition like polymer(s), drug(s), level of drug loading, surface area and dimensions of the implant etc. The rate can be episodic or periodic, or such that it is suitable for ocular and intra-ocular drug delivery having suitable release kinetics. The initial burst can be shortened by removing or rinsing the blend of drug at or near the surface of the implant or drug core or by coating the composition with a polymer that can be drug free or have a reduced drug content. In an embodiment, the implant can be loaded with a drug and premature or uncontrolled leakage of the drug is essentially avoided. Further, the drug can be embedded in a structure that regulates the release according to zero-order kinetic model. Such structures can be created using nano-technology and can include metal oxide or polymer matrices or other highly-controlled porous structures. The implant can also include small reservoir(s) of drug that can be opened such as by a laser or other energy source to apply a small electrical voltage to release the desired dose of the drug(s) on demand.

In addition, further control of the release of the one or more drugs or site-specific therapeutic agents can be achieved by formulating the drugs or site-specific therapeutic agents having one or more of a variety of features, including particle size, molecular weight, particle shape and particle thickness. Additionally, varying the ratio of the drug or site-specific therapeutic agent and drug release material can further control the release of the drug or site-specific therapeutic agent into the eye.

The drug-release material itself can dissolve, degrade, erode, absorb, or resorb over a period of time as well. In an embodiment, the drug-release material degrades from the interior volume of the implant over a period of at least 12 hours, at least 18 hours, at least 24 hours, at least 48 hours, at least 3 days, at least 7 days, at least 14 days, at least 30 days, at least 60 days, at least 90 days, at least 100 days, at least 120 days, at least 150 days, at least 180 days, at least 200 days, at least 250 days, at least 300 days, at least 350 days, at least 400 days, or even longer.

In an embodiment, the drug-release material can prevent substantial flow of fluid through the implant over a period of at least 12 hours, at least 18 hours, at least 24 hours, at least 48 hours, at least 3 days, at least 7 days, at least 14 days, at least 30 days, at least 60 days, at least 90 days, at least 100 days, at least 120 days, at least 150 days, at least 180 days, at least 200 days, at least 250 days, at least 300 days, at least 350 days, at least 400 days, or even longer.

In an embodiment, the implant 105 includes an interior volume that resembles a flow lumen having at least one inflow port at a first end and at least one outflow port at a second end. After a period of at least 12 hours, at least 18 hours, at least 24 hours, at least 48 hours, at least 3 days, at least 7 days, at least 14 days, at least 30 days, at least 60 days, at least 90 days, at least 100 days, at least 120 days, at least 150 days, at least 180 days, at least 200 days, at least 250 days, at least 300 days, at least 350 days, at least 400 days, or even longer, the drug-release material does not prevent substantial flow of fluid through the implant.

In an embodiment, the internal volume 135 of the implant 105 is filled with poly(lactic-co-glycolic acid) (PLGA) microspheres having a biodegradation rate such that after at least a period of days substantially all the drug has been eluted from the drug-release material and the drug-release material has degraded by at least a percent from the interior volume 135 of the implant 105. In an embodiment, substantially all the drug has eluted from the drug-release material in 180 days. In an embodiment, the drug-release material has degraded by at least 10%, 15%, 20%, 25%, 30%, 35%, 40%, 45%, 50%, 55%, 60%, 65%, 70%, 75%, 80%, 85% or more percent from the interior volume 135 of the implant 105.

The implants described herein can be manufactured as is known in the art. The implants can be machined or laser ablated from a unitary rod or block of stock material with the material subtracted or removed, leaving features behind. Alternatively, separate parts of the implant can be manufactured separately and assembled onto the implant. The implant can be manufactured by one or more injection molding or dip coating processes. The implants can be made of various materials, including, for example, polyimide, Nitinol, platinum, stainless steel, molybdenum, PVDF, silicone, or any other suitable polymer, metal, metal alloy, or ceramic biocompatible material or combinations thereof. Other materials of manufacture or materials with which the implant can be coated or manufactured entirely include Silicone, PTFE, ePTFE, differential fluoropolymer, FEP, FEP laminated into nodes of ePTFE, silver coatings (such as via a CVD process), gold, prolene/polyolefins, polypropylene, poly(methyl methacrylate) (PMMA), acrylic, PolyEthylene Terephthalate (PET), Polyethylene (PE), PLLA, and parylene.

The implants can be reinforced with polymer, Nitinol, or stainless steel braid or coiling or can be a co-extruded or laminated tube with one or more materials that provide acceptable flexibility and hoop strength for adequate lumen support and drainage through the lumen. The implant can alternately be manufactured of nylon (polyamide), PEEK, polysulfone, polyamideimides (PAI), polyether block amides (Pebax), polyurethanes, thermoplastic elastomers (Kraton, etc), and liquid crystal polymers. The implants can be at least partially manufactured of a mesh or braided structure formed of two or more interwoven strands, fibers, or threads of material. The interwoven strands can be arranged in a pattern that forms diamond-shaped holes or openings therebetween or openings of other shapes. The braided structure can be positioned over or otherwise combined with a solid tube. The implant can surround a core of drug that can be released through openings in the structure of the implant.

Embodiments in which the implant includes a drug-release material embedded within the interior volume can be prepared as is known in the art, for example, by simultaneously dissolving the polymer, drug, and, if present, optional component(s) in an organic solvent system capable of forming a homogenous solution of the polymer, drug, and optional component(s), solvent-casting the solution and then evaporating the solvent to leave behind a uniform, homogenous blend of polymer, drug and optional component(s).

The drug-polymer matrices can be fabricated by known methods (e.g., fiber spinning, electro-spinning, solvent casting, injection molding, thermoforming, etc.) to produce a desired structure for the implant. Depending on the thermal stability of the drug and the polymer, the articles can be shaped by conventional polymer-forming techniques such as extrusion, sheet extrusion, blown film extrusion, compression molding, injection molding, thermoforming, spray drying, injectable particle or microsphere suspension, and the like to form drug delivery implants. The drug-release material can be prepared by methods known in the art for forming biocompatible composites. In another embodiment, the drug can be incorporated into the structural material of the implant itself.

Embodiments in which the implant is coated with the drug-release material, the coatings can be spray-coated, dip coated, printed, or otherwise deposited can be prepared as is known in the art. The coating can be uniform or non-uniform such as dots or stripes or other pattern of material. The implant can include one or more layers of the coating. For example, a first or base layer can provide adhesion, a main layer can hold the drug to be eluted and a top coat can be used to slow down the release of the drug and extend its effect.

In some cases, it may be advantageous to have multiple main and top coat layers to provide varying drug release profiles. The implant can also include drug-release material on at least a surface of the implant that is in the form of a polymeric film.

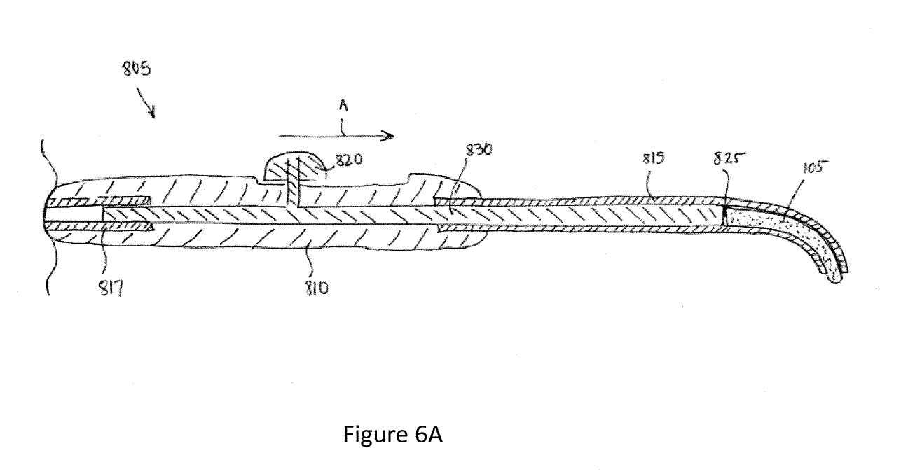

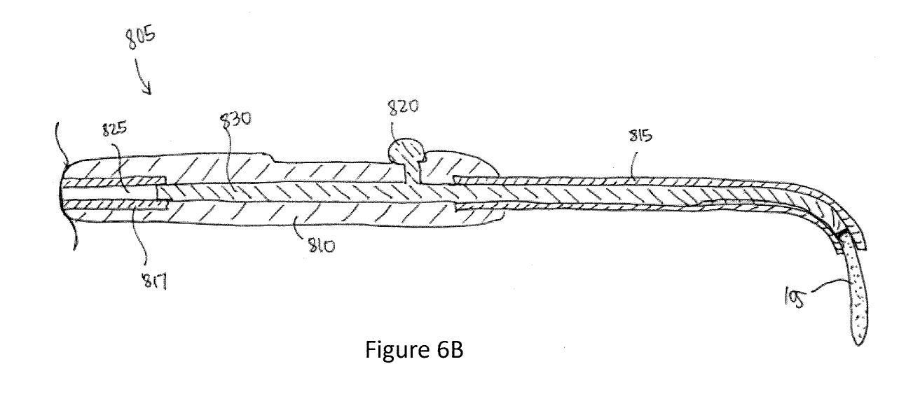

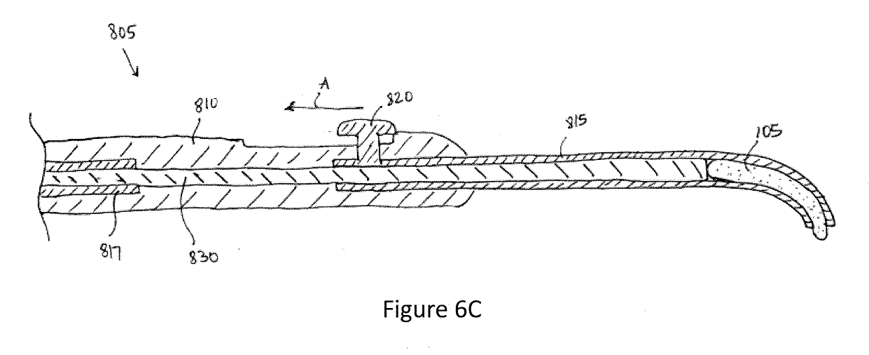

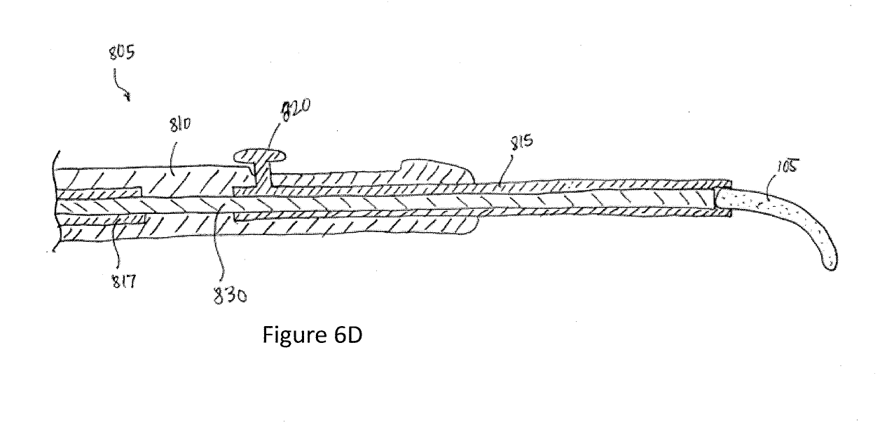

A variety of implantation systems can be used to deliver the drug delivery implant(s) described herein, such as the delivery devices described in U.S. Patent Publication number 2010-0137981, which is incorporated by reference herein in its entirety. FIGS. 6A-6D illustrate examples of an implantation system 805 that can be used to deliver at least some embodiments of the implants and release controlled drugs, including site-specific therapeutic agents, described herein. The implantation system 805 can generally include a proximal handle component 810 and a distal implantation component 815. The implantation component 815 is shown as being curved, but it should be appreciated that it could also be straight. The curvature of the implantation component 815 can vary. For example, the radius of curvature can be between about 3 mm to 50 mm and the curve can cover from 0 degrees to 180 degrees. In an embodiment, the radius of curvature can be around 12 mm.

The proximal handle component 810 can include an actuator 820 to control the release of the drug delivery implant(s) 105 from the elongate channel 825 of the implantation component 815 through which the implant 105 can be inserted longitudinally and into the target location in the eye. At least a portion of the distal region of the implant 105 can extend beyond the distal region of the implantation component 815 such that clogging is avoided during delivery. The implantation component 815 can also include a pusher 830 or other type of component that aids to release the implant 105 from the delivery device and into the eye. The pusher 830 can be coupled to the actuator 820 and act to push out the implant 105 from the distal end of the implantation component 815 upon sliding the actuator button 820 in a distal direction along arrow A (see FIGS. 6A-6B).

Alternatively, the actuator button 820 can be coupled to the implantation component 815 such that sliding the actuator button 820 proximally along arrow A retracts the implantation component 815 and releases the implant 105 (see FIGS. 6C-6D). In this embodiment, the pusher 830 remains fixed within the delivery device 805 such as at tube 817 (as opposed to being slidably coupled with tube 817 as shown in FIGS. 6A-6B) and prevents the implant 105 from traveling proximally with the implantation component 815 as it is retracted. It should be appreciated that although FIGS. 6A-6D illustrate the delivery of a single implant 105, more than one implant 105 can be delivered in the eye with one application of the delivery device 805. The one or more implants 105 can be delivered to a single location in the eye or spread out over multiple locations as described above.

During implantation, the distal region of the implantation component 815 can penetrate through a small, corneal incision to access the anterior chamber AC. In this regard, the single incision can be made in the eye, such as within the limbus of the cornea. In an embodiment, the incision is very close to the limbus, such as either at the level of the limbus or within 2 mm of the limbus in the clear cornea. The implantation component 815 can be used to make the incision or a separate cutting device can be used. For example, a knife-tipped device or diamond knife can be used to initially enter the cornea. A second device with a spatula tip can then be advanced over the knife tip wherein the plane of the spatula is positioned to coincide with the dissection plane.

The corneal incision can have a size that is sufficient to permit passage of the drug delivery implant(s) 105 in the implantation component 815 therethrough. In an embodiment, the incision is about 1 mm in size. In another embodiment, the incision is no greater than about 2.85 mm in size. In another embodiment, the incision is no greater than about 2.85 mm and is greater than about 1.5 mm. It has been observed that an incision of up to 2.85 mm is a self-sealing incision.

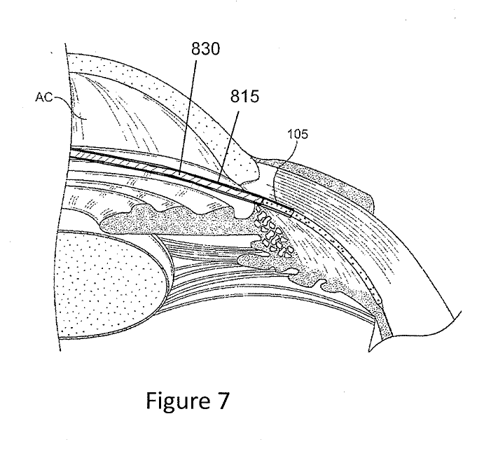

In one embodiment, after insertion through the incision the implantation component 815 can be advanced into the anterior chamber AC along a pathway that enables the implant 105 to be delivered from the anterior chamber toward the angle of the eye and into the supraciliary and/or the suprachoroidal space (see FIG. 7). With the implantation component 815 positioned for approach, the implantation component 815 can be advanced further into the eye towards the angle of the eye where the implantation component 815 can bluntly dissect and/or sharply penetrate tissues near the angle of the eye such that the supraciliary and/or suprachoroidal space can be entered. It should be appreciated that although FIG. 7 shows a single implant 105 being delivered into a location in the eye, more than one implant can be delivered using a single implantation component 815 and delivered during a single application using the delivery device 805.

The scleral spur is an anatomic landmark on the wall of the angle of the eye. The scleral spur is above the level of the iris but below the level of the trabecular meshwork. In some eyes, the scleral spur can be masked by the lower band of the pigmented trabecular meshwork and be directly behind it. The implantation component 815 can travel along a pathway that is toward the scleral spur such that the implantation component 815 passes near the scleral spur on the way to the suprachoroidal space. In an embodiment the implantation component 815 penetrates the scleral spur during delivery. In another embodiment, the implantation component 815 does not penetrate the scleral spur during delivery. The implantation component 815 can abut the scleral spur and move downward to dissect the tissue boundary between the sclera and the ciliary body, the dissection entry point starting just below the scleral spur near the iris root or the iris root portion of the ciliary body.

It should be appreciated that the pathway the implantation component 815 travels into the supraciliary and/or suprachoroidal space can vary. The implantation component 815 can bluntly dissect and/or sharply penetrate tissues near the angle of the eye such that the supraciliary and/or suprachoroidal space can be entered. In one example, the implantation component 815 penetrates the iris root. In another example, the implantation component 815 enters through a region of the ciliary body or the iris root part of the ciliary body near its tissue border with the scleral spur. In another example, the implantation component 815 can enter above or below the scleral spur. Another example, the implantation component 815 can enter through the trabecular meshwork.

The implantation component 815 can approach the angle from the same side of the anterior chamber as the deployment location such that the implantation component 815 does not have to be advanced across the iris. Alternately, the implantation component 815 can approach the angle from across the anterior chamber such that the implantation component 815 is advanced across the iris and/or the anterior chamber toward the opposite angle (see FIG. 7). The implantation component 815 can approach the angle along a variety of pathways. The implantation component 815 does not necessarily cross over the eye and does not intersect the center axis of the eye. In other words, the corneal incision and the location where the implantation component 815 enters the angle can be in the same quadrant. Also, the pathway of the device from the corneal incision to the angle ought not to pass through the centerline of the eye to avoid interfering with the pupil. The surgeon can rotate or reposition the handle of the delivery device 805 in order to obtain a proper approach trajectory for the implantation component 815.

The implantation component 815 with the implant 105 positioned therein can be advanced through to the supraciliary and/or suprachoroidal space. In one example, the implantation component 815 can be advanced such that it penetrates an area of fibrous attachment between the scleral spur and the ciliary body. This area of fibrous attachment can be approximately 1 mm in length. Once the distal tip of the implantation component 815 penetrates and is urged past this fibrous attachment region, it then more easily causes the sclera to peel away or otherwise separate from the choroid as it follows the inner curve of the sclera and forms the suprachoroidal space. The implantation component 815 can be continuously advanced into the eye. The dissection plane of the implantation component 815 follows the curve of the inner scleral wall such that it bluntly dissects the boundary between tissue layers of the scleral spur and the ciliary body. A combination of the tip shape, material, material properties, diameter, flexibility, compliance, coatings, pre-curvature etc. of the implantation component 815 make it more inclined to follow an implantation pathway that mirrors the curvature of the inner wall of the sclera and between tissue layers such as the sclera and choroid. The dynamics of the implantation component is described in more detail in U.S. Patent Publication number 2010-0137981, which is incorporated by reference herein in its entirety.

As described above, the implant 105 can be positioned within a variety of regions of the eye using the delivery device 805. For example, the implant 105 can be positioned within the supraciliary space, the suprachoroidal space or other locations deeper in the eye such as toward the back of the eye. Other locations for implant 105 are also possible. It should also be appreciated that multiple depositions of a plurality of drug delivery implants 105 can be performed in various zones of the eye during a single approach and dissection using the delivery device 805.

In some embodiments, once the implant 105 is released within the eye the drug-release material can slowly elute drug such that the implant 105 delivers therapy to the eye in a time-release manner. After a period of time, the drug is largely eluted from the drug-release material. The drug-release material can also degrade over time leaving an open interior volume of the implant 105. The implant 105 can be left in place such that the open interior volume can provide a flow channel for aqueous to exit the anterior chamber. Alternative, the implant 105 can be recharged with drug-release material or the implant 105 can be removed from the eye either by manual removal or by biodegradation of the implant 105 within the eye.

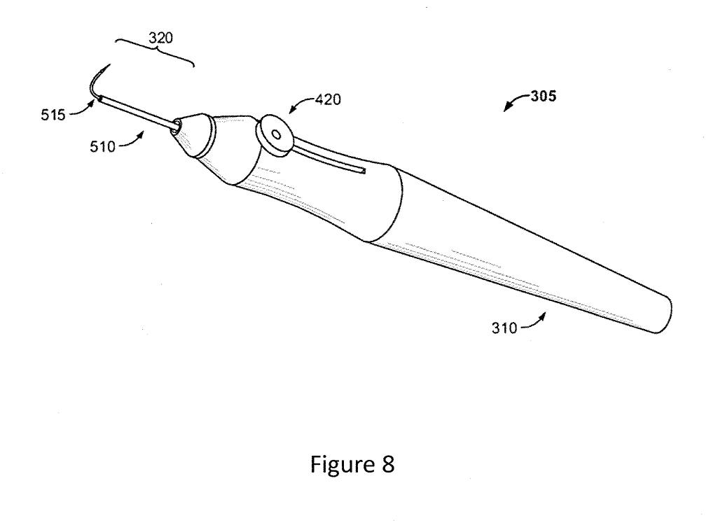

The implants can be delivered pre-loaded with the drug-release material, including site-specific therapeutic agents, within the interior volume or the implants can be filled with the drug-release material upon delivery into the eye. FIGS. 8 and 9A-9D illustrate examples of an implantation system 305 that can be used to deliver an implant 605 that can be filled with a drug-release material 610, including site-specific therapeutic agents, upon delivery into the eye. It should be appreciated that these implantation systems 305 are for illustration and that variations in the structure, shape and actuation of the implantation system 305 are possible.

The implantation system 305 can generally include a proximal handle component 310 and a distal implantation component 320. The implantation component 320 is shown as being curved, but it should be appreciated it could also be straight. The curvature of the implantation component 320 can vary. For example, the radius of curvature can be between about 3 mm to 50 mm and the curve can cover from 0 degrees to 180 degrees. In an embodiment, the radius of curvature can be around 12 mm. The proximal handle component 310 can include an actuator 420 to control the release of an implant from the implantation component 320 into the target location in the eye.

The delivery component 320 can include an elongate applier 515 that can insert longitudinally through the implant 605 and a sheath 510 that can be positioned axially over the applier 515. The sheath 510 can aid in the release of the implant 605 from the delivery component 320 into the target location in the eye. The actuator 420 can be used to control the applier 515 and/or the sheath 510. For example, the sheath 510 can be urged in a distal direction relative to the applier 515 to push the implant 605 off the distal end of the applier 515.

Alternately, the sheath 510 can be fixed relative to the handle component 310. In this embodiment, the sheath 510 can act as a stopper that impedes the implant 105 from moving in a proximal direction as the applier 515 is withdrawn proximally from the implant 605 upon actuation of the actuator 420. The applier 515 can be extended distally relative to the sheath 310. Movement of the actuator 420, such as in the proximal direction, can cause the applier 515 to slide proximally into the sheath 510. This effectively pushes the implant 605 off the distal end of the applier 515 and releases the implant 605 in a controlled fashion such that the target positioning of the implant 605 within the suprachoroidal space is maintained.

As the implant 605 is released, the applier 515 is withdrawn from the internal volume of the implant 605. A drug-release material, including a site-specific therapeutic agent, 610 can be injected into the internal volume 635 of the implant 605 as the applier 515 is withdrawn. FIGS. 9A-9D show the interior volume of an implant 605 being injected with a drug-release material 610 as the applier 515 is withdrawn from the implant 605. In this embodiment, the applier 515 can include a bore 620 through which the drug-release material 610 can be injected into the internal volume 635 of the implant 605.

The drug-release material 610 can be injected from a larger volume source through a catheter coupled to the delivery instrument using positive pressure delivered to the delivery instrument such as by a pump or a syringe or other device configured to inject material through the applier 515. The drug-release material 610 can include a viscoelastic material with a drug, such as a site-specific therapeutic agent, incorporated into it as described herein. It should be appreciated that other flowable materials besides drug-release material can be injected using a positive pressure source.

In addition, for example, the site-specific therapeutic agents can include anti-fibrotic and anti-inflammatory agents. Furthermore, various ratios of the drugs or site-specific therapeutic agents with one or more drug release materials, including viscoelastic, can be injected for releasing the drugs or site-specific therapeutic agents in a variety of release profiles.

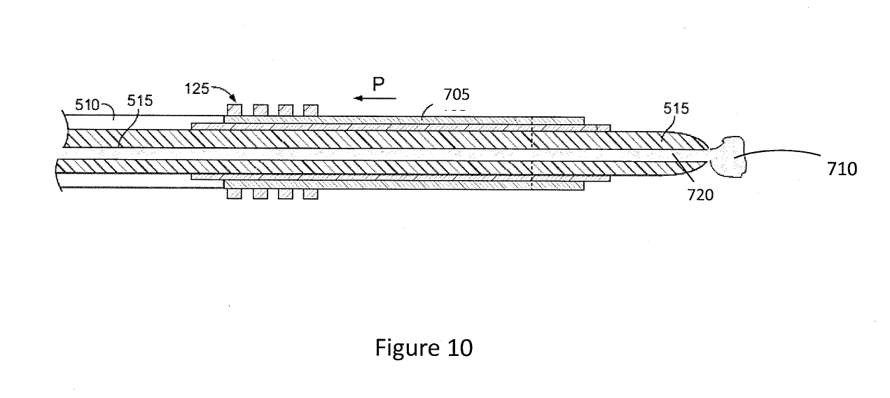

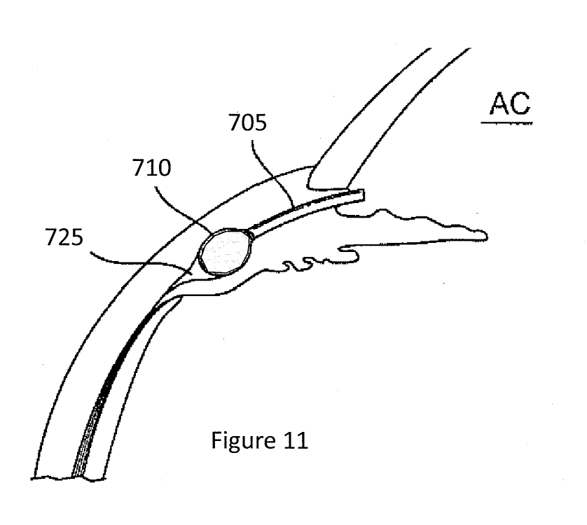

In an alternative embodiment shown in FIGS. 10-11, a distal deposition 710 of material can be deposited at or near the distal end of the implant 705 prior to and/or during withdrawal of the applier 515 from the bore. The distal deposition 710 can be used to hydro-dissect a space between tissue layers, for example by viscodissection, to further expand an area or create a “lake” 725 between the tissue layers at or near the distal end of the implant 705, such that the layers are no longer strongly adhered and/or the tissues apposed. The lake 725 can be entirely enclosed by tissue and allow for the accumulation of fluid between the tissues.

In an embodiment, the distal deposition 710 can be flowed into the suprachoroidal space, such as through a delivery instrument as shown in FIG. 10. In addition, the delivery instrument can be coupled to a positive pressure source such as a pump or syringe for injecting the distal deposition 710 from a source, as discussed above. The distal deposition 710 is flowed into the eye with a pressure sufficient to form a dissection plane within the suprachoroidal space such that the fluid then accumulates within the suprachoroidal space so as to form a lake 725.

The distal deposition 710 can be formed within the suprachoroidal space such that the implant 705 is positioned with its proximal end in communication with the anterior chamber AC and its distal end positioned such that the distal deposition 710 can be flowed into the suprachoroidal space. It should be appreciated that the distal deposition 710 of material may or may not also fill the interior volume of the implant 705.

The distal deposition 710 can be a viscoelastic material, such as hyaluronic acid, that is loaded with a drug or other active agent from which the drug or other active agent can elute over time. It should also be appreciated that the distal deposition 710 need not be loaded with a drug or an active agent. The viscoelastic material can allow for the diffusion of fluid therethrough. In this embodiment, aqueous fluid from the anterior chamber AC can flow through the implant 705 as well as through and around the distal deposition 710 forming the lake 725.

The distal deposition 710 can be protected from the aqueous fluid of the anterior chamber AC such that the rate of degradation of the drug-release material can be extended. In an embodiment, the distal deposition 710 deposited near the distal end of the implant 705 is protected from exposure to the aqueous and has a degradation rate that is at least, and preferably longer than 12 hours. The deposited material can result in the formation of a void between the tissue layers that remains after degradation of the material. The lake creates a volume where the tissues can be permanently detached or weakly adhesed and scarring together is avoided.

The size and volume of the lake formed by the distal deposition 710 can influence the flow of fluid out of the anterior chamber. For example, if the distal deposition 710 near the distal end of the implant 705 is too large, the flow of aqueous from the anterior chamber can be too great and result in hypotony. If the distal deposition 710 near the distal end of the implant 705 is too small, the flow of aqueous from the anterior chamber can be too minor and intraocular pressure unimproved. Injection of varying volumes of material near the distal end of the implant 705 can be used as a method of controlling flow out of the anterior chamber and customized for a particular patient and the pressure relief needed to treat the disease.

The hydrophobicity and hydrophilicity of the material used to create the lake or distal deposition 710 can also impact the amount of fluid flow through the implant from the anterior chamber. Viscoelastic compositions such as a viscoelastic preparation of sodium hyaluronate, which is a hydrophilic polymer. The material used to create the lake can also be altered to obtain a customized residence time. For example, adding cross-links to the material can increase the overall residence time of the material within the lake.

The devices described herein can be used to deliver essentially any active substance. As used herein, “substance,” “drug” or “therapeutic” is an agent or agents that ameliorate the symptoms of a disease or disorder or ameliorate the disease or disorder including, for example, small molecule drugs, proteins, nucleic acids, polysaccharides, and biologics or combination thereof. Therapeutic agent, therapeutic compound, therapeutic regimen, or chemotherapeutic include conventional drugs and drug therapies, including vaccines, which are known to those skilled in the art. Therapeutic agents include, but are not limited to, moieties that inhibit cell growth or promote cell death, that can be activated to inhibit cell growth or promote cell death, or that activate another agent to inhibit cell growth or promote cell death. Optionally, the therapeutic agent can exhibit or manifest additional properties, such as, properties that permit its use as an imaging agent, as described elsewhere herein. Additionally, therapeutic agents can be site-specific such that they are released in or adjacent a part of the eye which is intended to be directly affected by the therapeutic agent.

Exemplary therapeutic agents include, for example, cytokines, growth factors, proteins, peptides or peptidomimetics, bioactive agents, anti-fibrotics, anti-inflammatory, photosensitizing agents, radionuclides, toxins, anti-metabolites, signaling modulators, anti-cancer antibiotics, anti-cancer antibodies, angiogenesis inhibitors, radiation therapy, chemotherapeutic compounds or a combination thereof. The drug may be any agent capable of providing a therapeutic benefit. In an embodiment, the drug is a known drug, or drug combination, effective for treating diseases and disorders of the eye. In non-limiting, exemplary embodiments, the drug is an anti-infective agent (e.g., an antibiotic or antifungal agent), an anesthetic agent, an anti-VEGF agent, an anti-inflammatory agent, a biological agent (such as RNA), an intraocular pressure reducing agent (i.e., a glaucoma drug), or a combination thereof. Non-limiting examples of drugs are provided below.

A variety of therapeutic agents can be delivered using the drug delivery implants described herein, including: anesthetics, analgesics, cell transport/mobility impending agents such as colchicine, vincristine, cytochalasin B and related compounds; antiglaucoma drugs including beta-blockers such as timolol, betaxolol, atenolol, and prostaglandins, lipid-receptor agonists or prostaglandin analogues such as bimatoprost, travoprost, latanoprost, unoprostone etc; alpha-adrenergic agonists, brimonidine or dipivefrine, carbonic anhydrase inhibitors such as acetazolamide, methazolamide, dichlorphenamide, diamox; and neuroprotectants such as nimodipine and related compounds.

Additional examples include antibiotics such as tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole and sulfisoxazole; anti-fungal agents such as fluconazole, nitrofurazone, amphotericin B, ketoconazole, and related compounds; anti-viral agents such as trifluorothymidine, acyclovir, ganciclovir, DDI, AZT, foscamet, vidarabine, trifluorouridine, idoxuridine, ribavirin, protease inhibitors and anti-cytomegalovirus agents; antiallergenics such as methapyriline; chlorpheniramine, pyrilamine and prophenpyridamine; anti-inflammatories such as hydrocortisone, dexamethasone, fluocinolone, prednisone, prednisolone, methylprednisolone, fluorometholone, betamethasone and triamcinolone; decongestants such as phenylephrine, naphazoline, and tetrahydrazoline; miotics, muscarinics and anti-cholinesterases such as pilocarpine, carbachol, di-isopropyl fluorophosphate, phospholine iodine, and demecarium bromide; mydriatics such as atropine sulfate, cyclopentolate, homatropine, scopolamine, tropicamide, eucatropine; sympathomimetics such as epinephrine and vasoconstrictors and vasodilators; Ranibizumab, Bevacizamab, and Triamcinolone.

Anti-inflammatories, such as non-steroidal anti-inflammatories (NSAIDs) may also be delivered, such as cyclooxygenase-1 (COX-1) inhibitors (e.g., acetylsalicylic acid, for example ASPIRIN from Bayer AG, Leverkusen, Germany; ibuprofen, for example ADVIL from Wyeth, Collegeville, Pa.; indomethacin; mefenamic acid), COX-2 inhibitors (CELEBREX from Pharmacia Corp., Peapack, N.J.; COX-1 inhibitors), including a prodrug NEPAFENAC; immunosuppressive agents, for example Sirolimus (RAPAMUNE, from Wyeth, Collegeville, Pa.), or matrix metalloproteinase (MMP) inhibitors (e.g., tetracycline and tetracycline derivatives) that act early within the pathways of an inflammatory response. Anticlotting agents such as heparin, anti-fibrinogen, anti-fibrotics, fibrinolysin, anti clotting activase, etc., can also be delivered.

Antidiabetic agents that may be delivered using the disclosed implants include acetohexamide, chlorpropamide, glipizide, glyburide, tolazamide, tolbutamide, insulin, aldose reductase inhibitors, etc. Some examples of anti-cancer agents include 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil mustard, vinblastine, vincristine and vindesine.

Hormones, peptides, steroids, nucleic acids, saccharides, lipids, glycolipids, glycoproteins, and other macromolecules can be delivered using the present implants. Examples include: endocrine hormones such as pituitary, insulin, insulin-related growth factor, thyroid, growth hormones; heat shock proteins; immunological response modifiers such as muramyl dipeptide, cyclosporins, interferons (including α, β, and γ interferons), interleukin-2, cytokines, FK506 (an epoxy-pyrido-oxaazcyclotricosine-tetrone, also known as Tacrolimus), tumor necrosis factor, pentostatin, thymopentin, transforming factor beta2, erythropoetin; antineogenesis proteins (e.g., anti-VEGF, Interferons), among others and anticlotting agents including anticlotting activase. Further examples of macromolecules that can be delivered include monoclonal antibodies, brain nerve growth factor (BNGF), ciliary nerve growth factor (CNGF), vascular endothelial growth factor (VEGF), and monoclonal antibodies directed against such growth factors. Additional examples of immunomodulators include tumor necrosis factor inhibitors such as thalidomide.

In addition, nucleic acids can also be delivered wherein the nucleic acid may be expressed to produce a protein that may have a variety of pharmacological, physiological or immunological activities. Thus, the above list of drugs is not meant to be exhaustive. A wide variety of drugs or agents may be used in the present invention, without restriction on molecular weight, etc.

Other agents include anti-coagulant, an anti-proliferative, imidazole antiproliferative agent, a quinoxaline, a phosphonylmethoxyalkyl nucleotide analog, a potassium channel blocker, and/or a synthetic oligonucleotide, 5-[1-hydroxy-2-[2-(2-methoxyphenoxyl)ethylamino]ethyl]-2-methylbenzenesulfonamide, a guanylate cyclase inhibitor, such as methylene blue, butylated hydroxyanisole, and/or N-methylhydroxylamine, 2-(4-methylaminobutoxy)diphenylmethane, apraclonidine, a cloprostenol analog or a fluprostenol analog, a crosslinked carboxy-containing polymer, a sugar, and water, a non-corneotoxic serine-threonine kinase inhibitor, a nonsteroidal glucocorticoid antagonist, miotics (e.g., pilocarpine, carbachol, and acetylcholinesterase inhibitors), sympathomimetics (e.g., epinephrine and dipivalylepinephxine), beta-blockers (e.g., betaxolol, levobunolol and timolol), carbonic anhydrase inhibitors (e.g., acetazolamide, methazolamide and ethoxzolamide), and prostaglandins (e.g., metabolite derivatives of arachidonic acid, or any combination thereof.

Additional examples of beneficial drugs that may be employed in the present invention and the specific conditions to be treated or prevented are disclosed in Remington, supra; The Pharmacological Basis of Therapeutics, by Goodman and Gilman, 19th edition, published by the MacMillan Company, London; and The Merck Index, 13th Edition, 1998, published by Merck & Co., Rahway, N.J., which is incorporated herein by reference.

It should be appreciated that other ocular conditions besides glaucoma can be treated with the drug delivery implants described herein. For example, the compositions and methods disclosed herein can be used to treat a variety of diseases and/or conditions, for example: eye infections (including, but not limited to, infections of the skin, eyelids, conjunctivae, and/or lacrimal excretory system), orbital cellulitis, dacryoadenitis, hordeolum, blepharitis, conjunctivitis, keratitis, corneal infiltrates, ulcers, endophthalmitis, panophthalmitis, viral keratitis, fungal keratitis herpes zoster ophthalmicus, viral conjunctivitis, viral retinitis, uveitis, strabismus, retinal necrosis, retinal disease, vitreoretinopathy, diabetic retinopathy, cytomegalovirus retinitis, cystoids macular edema, herpes simplex viral and adenoviral injections, scleritis, mucormycosis, canaliculitis, acanthamoeba keratitis, toxoplasmosis, giardiasis, leishmanisis, malaria, helminth infection, etc. It also should be appreciated that medical conditions besides ocular conditions can be treated with the drug delivery implants described herein. For example, the implants can deliver drugs for the treatment of inflammation, infection, cancerous growth. It should also be appreciated that any number of drug combinations can be delivered using any of the implants described herein.

The present disclosure includes a variety of site-specific therapeutic agents and their delivery for providing various release profiles of the site-specific therapeutic agents to one or more parts of the eye. The various release profiles can allow an effective amount of site-specific therapeutic agent to be released into the eye over an extended period of time which can result in improved treatment of the eye. In addition, the site-specific therapeutic agents can be delivered to the eye either directly or via an implant loaded with the site-specific therapeutic agents.

The site-specific therapeutic agents can be contained within a releasing agent which can assist in characterizing the release profile of the site-specific therapeutic agents into the eye. For example, a releasing agent, such as a viscoelastic, can be mixed with one or more site-specific therapeutic agents which can then be delivered either directly into the eye or into a part of an optical implant which can then be implanted into the eye. The site-specific therapeutic agents can then release from the releasing agent into the eye over one or more releasing profiles for providing treatment to the eye, such as in order to prevent fibrotic and inflammatory responses due to the placement of an implant, surgical procedure or disease of the eye.

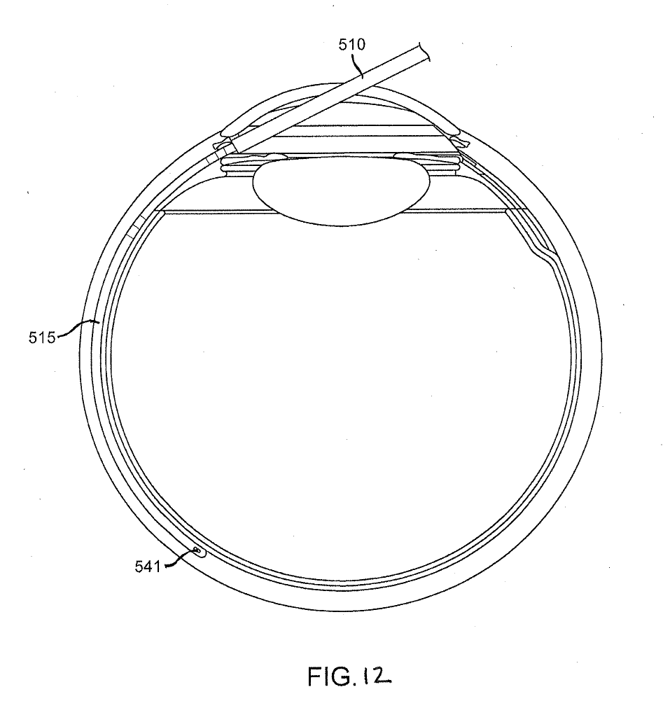

As shown in FIG. 12, a hollow guidewire 515 having at least one through hole 541 can deliver site-specific therapeutic agents contained within a releasing agent to the eye. For example, the guidewire 515 can be inserted through a corneal incision and inserted into the supraciliary space via an ab-interno procedure. One or more site-specific therapeutic agents mixed within the releasing agent can then be delivered through the at least one through hole 541 within or adjacent either the suprachroroidal space or supraciliary space.

Alternatively or in addition, the guidewire 515 can be further advanced until at least one through hole 541 is positioned within or adjacent a sub-retinal space, as shown in FIG. 12. The site-specific therapeutic agents mixed within the releasing agent can then be delivered through the at least one through hole 541. Once delivered, the site-specific therapeutic agents can release into the eye, including various tissue structures of the eye, over a period of time, as defined by one or more releasing profiles.

Once the site-specific therapeutic agents mixed within the releasing agent has been delivered to one or more parts of the eye, the guidewire 515, or any of a variety of fluid delivery devices, can then be removed leaving the site-specific therapeutic agents to release from the releasing agent over one or more releasing profiles. As discussed above, releasing the site-specific therapeutic agents over an extended period of time, as defined by the releasing profiles, can allow the site-specific therapeutic agents to be more effective, such as preventing fibrosis and inflammation of the eye at least at or near where the site-specific therapeutic agents is released within the eye.

The guidewire 515 or fluid delivery device can be part of an implant delivery system, such as the implantation system 805 described herein. Alternatively or in addition, the guidewire 515 or fluid delivery device can be configured solely for the delivery of fluid, such as site-specific therapeutic agents, into the eye.

As discussed above, the site-specific therapeutic agents can be mixed with a releasing agent and can be either delivered directly into the eye or delivered into an ocular implant. In some embodiments, the site-specific therapeutic agents mixed within a releasing agent can be delivered into an implant which has been implanted within the eye. Alternatively or in addition, the site-specific therapeutic agents can be delivered into the implant prior to implantation of the implant into the eye. In either case, the implant can assist in allowing the site-specific therapeutic agents mixed with the releasing agent to release into the eye at or adjacent the implantation site of the implant.

In some embodiments, the implant can provide additional characterization of the releasing profiles of the site-specific therapeutic agents. For example, some materials of the implant can further slow down or speed up the release of the site-specific therapeutic agents into the eye.

The release profile of any one of the site-specific therapeutic agents into the eye can be affected by at least the composition of the site-specific therapeutic agent relative to the releasing agent which the site-specific therapeutic agent is mixed with. In addition, the formulation and composition of one or more site-specific therapeutic agents mixed with one or more releasing agents can be customized for a variety of treatments of the eye.

For example, the site-specific therapeutic agents can be mixed with a releasing agent in a variety of ratios. For example, site-specific therapeutic agents, including anti-fibrotics such as 5-Fluorouracil (5FU) and Mitomycin-C (MMC), can be mixed with a releasing agent, including viscoelastics such as hyaluronic acid (HA), prior to injection into either the eye or implant. Various ratios between the anti-fibrotics and the viscoelastics can be made in order to create a desired release profile of the site-specific therapeutic agent, such as the anti-fibrotics, into the eye. Although anti-fibrotics are used an example, any number of site-specific therapeutic agents and drugs can be used with the releasing agent in order to provide a desired therapeutic effect over a desired time period.

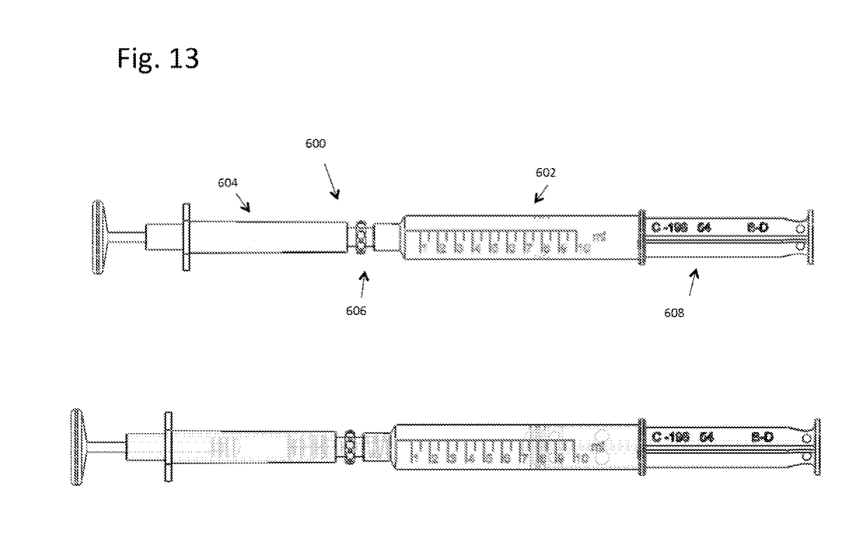

FIG. 13 shows an embodiment of a dual syringe apparatus 600 which can be used to mix one or more site-specific therapeutic agents with one or more releasing agents. For example, a first syringe 602 of the dual syringe apparatus 600 can be filled with a volume of at least one site-specific therapeutic agent. In addition, a second syringe 604 of the dual syringe apparatus 600 can be filled with a volume of at least one releasing agent. The first syringe 602 can be coupled to the second syringe 604 with a coupling element 606 in order to provide fluid communication between the first syringe 602 and second syringe 604, as shown in FIG. 13. A user can alternate pushing a plunger 608 associated with either the first syringe 602 or second syringe 604 such that the site-specific therapeutic agent and release agent are exchanged back and forth between the first syringe 602 and second syringe 604 which can effectively mix the site-specific therapeutic agent and release agent. For example, once the therapeutic agent and releasing agent have been mixed, the mixed solution can be either delivered directly into the eye or into an implant for delivery into the eye.

In addition, mixing the site-specific therapeutic agent and release agent with the dual syringe apparatus 600 can assist in creating a homogenous distribution of the site-specific therapeutic agent within the release agent. For example, homogenous distribution of the site-specific therapeutic agent within the release agent can allow the site-specific therapeutic agent to more effectively follow a desired release profile. In some embodiments, the site-specific therapeutic agent and release agent are exchanged at least approximately 4 times between the first syringe 602 and second syringe 604 in order to achieve homogeneity. In addition, site-specific therapeutic agent and release agent can be exchanged at least approximately 8 times, 12 times, 15 times, 20 times, or more. The number of exchanges of fluid between the first syringe 602 and the second syringe 604 in order to achieve homogeneity can depend on a variety of factors, including the type and volume of site-specific therapeutic agent and release agents being mixed, and the size syringes being used.

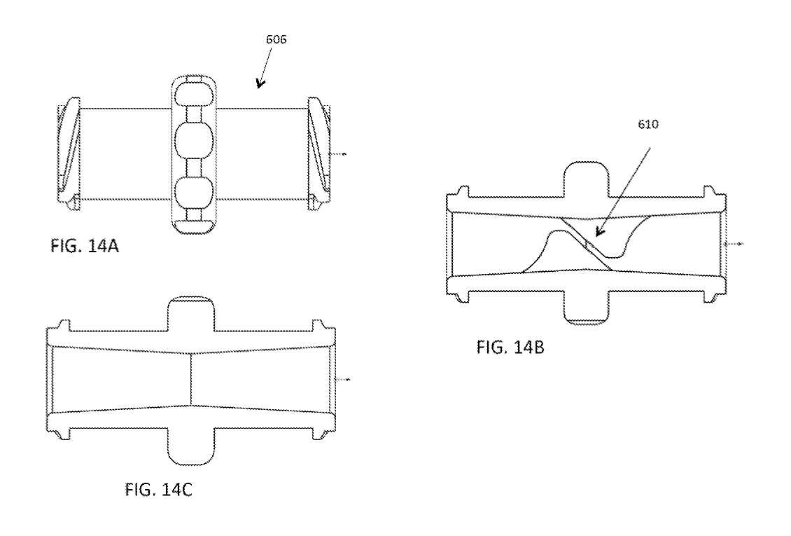

As shown in FIGS. 14B, and 14C, the coupling element 606 of the dual syringe apparatus 600 may include a straight bore channel that extends therethrough and provides an inner profile for fluidly coupling the syringes. This inner profile of the coupling element 606 may be shaped to provide a low amount of resistance to the flow of the mixture from one syringe to the other. Alternatively, the coupling element 606 may additionally include a mixing geometry 610 along the inner profile which is shaped further promote the mixing of the therapeutic agent and the release agent such that potentially fewer transfers from one syringe to the other syringe are required. The mixing geometry may vary.

In an embodiment shown in FIG. 14B, the mixing geometry 610 may include a swirl or corkscrew profile that is constricts and rotates the flow of the mixture for a greater distance. Alternatively, a straight profile with a constricted opening may be used as shown in FIG. 14C. Alternatively, a flat shear plane may be used to alter the properties of the hyaluronic acid viscoelastic release agent. For example, some hyaluronic acids such as Healon5 can have a variety of mechanical properties as different shear states such as high shear states. These high shear states may improve mixing between the therapeutic agent and the hyaluronic acid that may be desired. Any other number of mixing geometries may be considered.

In some embodiments the release agent and the therapeutic agent may be supplied in pre-packaged containers. For example, hyaluronic acid is commonly available for ophthalmology surgeries and supplied in vials with kits which include a Luer connection or other type of connection. Additionally therapeutic agents may be supplied in similar syringe kits with Luer connections. Alternatively, the therapeutic agent may be mixed with a dilutive agent and filled into the first syringe 602. The connecting element 606 may include a female Luer connection on both sides such that the first syringe 602 and the second syringe 604 may be easily connected to the connecting element 606. Alternatively, any other number of connection methods may be used such as push-to-connect fittings or any other suitable fitting method which connect the first syringe 602 to the second syringe 604. The connecting element 606 may be formed of any suitable medical grade material such as a medical grade plastic such as polycarbonate, nylon, polypropylene or any other suitable medical grade plastic. Alternatively, the connecting element 606 may include multiple components that are fused together to create the fluid communication between the first syringe 602 and the second syringe 604.

In some embodiments, either 5FU or MMC can be mixed with HA such that the ratio between the therapeutic agent and the viscoelastic is approximately 1:1, 1:2, 1:3, 2:3, 1:6, 1:4, etc. The ratio of the site-specific therapeutic agent to therapeutic agent can assist in characterizing the delivery profile of the site-specific therapeutic agent to one or more parts of the eye. Therefore, any number of ratios of the site-specific therapeutic agent to the releasing agent can be made in order to achieve a desired delivery profile of the one or more site-specific therapeutic agent. The therapeutic agent may be considered the material in the first syringe 602 which may be diluted with a diluting agent.

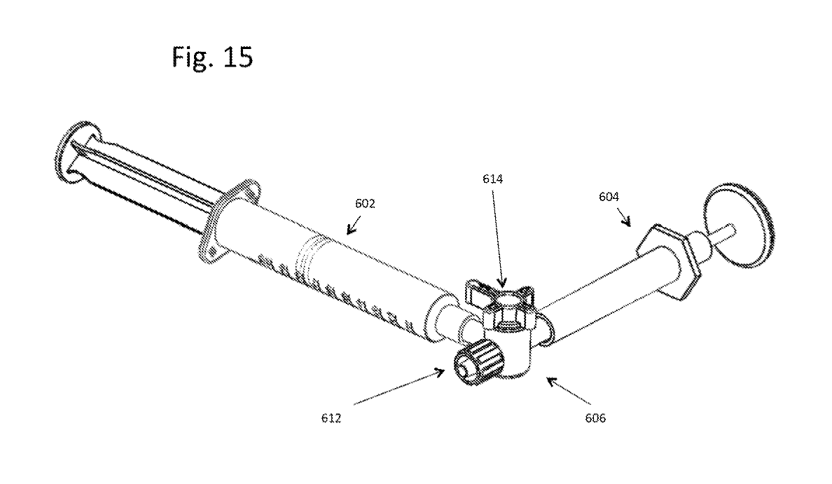

The connecting element 606 can include two outlets and inlets as shown in FIG. 13. Alternatively, as shown in FIG. 15, the connecting element 606 can have multiple inlets and outlets which are selectable by the user. A dispensing outlet 612 may exist which can be connected to a delivery device capable of delivering the mixture of the first syringe and the second syringe to the eye or ocular implant. In this embodiment, the user may use the control valve 614 on the connecting element 606 to fluidly connect only the first syringe 602 and second syringe 604. The user may then push on at least one of the plungers as described to transfer the contents of one of the syringes to the other. After a certain number of transfers the therapeutic agent and the release agent may be considered sufficiently homogenous the user may desire to deliver the mixture to the eye or an ocular implant. At this time or prior, the user may connect the connecting element 606 shown in FIG. 14 to a delivery device at the dispensing outlet. The user may then rotate the control valve such that the syringe with the mixture may be fluidly connected to the delivery device. The user may then delivery the mixture to the eye or ocular implant. In other embodiments, the connecting element 606 may include multiple outlets and inlets such that multiple syringes with therapeutic agents and releasing agents may be connected.

The therapeutic agent releasing profile can include the volume of therapeutic agent released over time, which may vary over the course of releasing the therapeutic agent. The time which therapeutic agent is released can vary and can depend on a number of factors, including type and volume of therapeutic agent being released into the eye and type of treatment being sought. At least some benefits of controlling the release of site-specific therapeutic agents within the eye, including releasing the therapeutic agents along a releasing profile can include improved accurate dosing of the site-specific therapeutic agents to one or more parts of the eye, minimizing side effects due to improper application of site-specific therapeutic agents, improved sustainability and control of therapeutic agents with the eye, increased bioavailability of the therapeutic agents, and improved patient compliance.

Delivery of the site-specific therapeutic agents mixed with one or more releasing agents can be achieved in a variety of ways including sub-conjunctival injection or implant, intravitreal implant, contact lenses, punctual plugs, collagen shield, shunts, stents, ocular iontophoresis cannulation, microneedles which can deliver fluid directly to the suprachoroidal or supraciliary space, liposome injections, niosome injections, nanoparticles or microparticles loaded with site-specific therapeutic agents, and hyaluronic acid loaded with site-specific therapeutic agents.

At least some site-specific therapeutic agents can include antibiotics, immunomodulators, H1 receptor antagonists, anti-fibrotics, anti-glaucoma, anti-inflammatory, anti-viral, anti-fungal, and any other drug or therapeutic agent disclosed herein. Some ocular diseases which can be treated with the controlled release of the one or more site-specific therapeutic agents can include at least age-related Amacular degeneration, allergies, angiogenesis, capillary non-perfusion, cataracts, conjunctivitis, corneal wound healing, diabetic macular edema, diabetic retinopathy, dry eye syndrome, edema, glaucoma-ocular hypertension, gougerot-sjogren syndrome, keratoconjunctivitis sicca, ocular neurodegeneration, ocular neurovascularization, ocular pain, retinal vein occlusion, retinitis pigmentosa, rosacea, trachoma, uveitis, and visual defects.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A method of delivering a therapeutic agent into an eye, comprising:
coupling a first syringe of a multi-syringe apparatus containing the therapeutic agent to a second syringe of the multi-syringe apparatus containing a releasing agent;
mixing the therapeutic agent with the releasing agent by pushing on at least one plunger of the multi-syringe apparatus; and
dispensing the therapeutic agent mixed with the releasing agent into at least one of a part of the eye or an ocular implant.

2. The method of claim 1 wherein the therapeutic agent is an antiproliferative drug.

3. The method of claim 2 wherein the antiproliferative drug is mitomycin C.

4. The method of claim 2 wherein the antiproliferative drug is 5-fluorouracil.

5. The method of claim 1 wherein the releasing agent is a hyaluronic acid.

6. The method of claim 1 wherein the ratio of therapeutic agent to releasing agent is between 1:1 and 1:8.

7. A device for delivering a therapeutic agent into an eye, comprising:
a first syringe containing the therapeutic agent;
a second syringe containing a releasing agent;
a coupling element configured to fluidly connect the first syringe to the second syringe;
a plunger in at least one of the syringes that is configured to push a contents of one syringe into another syringe.

8. The device of claim 7, wherein the coupling element has a fluid path geometries that promote the mixing of the therapeutic agent and release agent.

9. The device of claim 7 wherein the coupling element is configured to connect to a delivery device capable of dispensing the therapeutic agent mixed with the release agent into at least one of a part of the eye or an ocular implant.
